(19) United States
(12) Patent Application Publication
(10) Pub. No.: US 2014/0330128 A1
(43) Pub. Date: Nov. 6, 2014

(54) SIGNAL PROCESSING APPARATUS AND METHOD

(71) Applicant: Sony Corporation, Tokyo (JP)

(72) Inventor: Tatsumi Sakaguchi, Kanagawa (JP)

(73) Assignee: Sony Corporation, Tokyo (JP)

(21) Appl. No.: 14/359,953

(22) PCT Filed: Nov. 22, 2012
(86) PCT No.: PCT/JP2012/080257

§ 371 Date: May 22, 2014

(30) Foreign Application Priority Data
Nov. 30, 2011 (JP) 2011-261332

(51) Int. Cl. A61B 8/00; A61B 8/08; G06F 19/00

(52) U.S. Cl. CPC A61B 8/4245; G06F 19/3406; A61B 8/4494; A61B 8/52; A61B 8/54; A61B 8/4272; A61B 8/4461
USPC 600/472; 600/459

(57) ABSTRACT

The present disclosure relates to a signal processing apparatus and a signal processing method for increasing the precision of probe movement calculations.

An A array transducer is a one-dimensional array transducer that is a conventional array transducer. A B array transducer and a C array transducer are connected to both ends of the short side of the A array transducer (the right and left ends in the drawing), so that the array direction of the respective transducers in the A array transducer is perpendicular to the array direction of the respective transducers in the B array transducer and the C array transducer. The present disclosure can be applied to a signal processing apparatus that generates an ultrasound image from signals supplied from a probe that captures ultrasound images, and displays the generated ultrasound image.

### TECHNICAL FIELD

The present disclosure relates to signal processing apparatuses and methods, and more particularly, to a signal processing apparatus and a signal processing method for increasing the precision of probe movement calculations.

### BACKGROUND ART

In an ultrasound diagnostic apparatus that captures ultrasound images, detection of probe movement serves an important role for computer-aided diagnoses, measurement of tissue forms and behaviors, generation of panoramic images, processing of 3D reconstruction, or the like.

As for detection of probe movement, Patent Document 1 discloses a method of forming two scanning planes with a two-dimensional probe, and conducting detection of probe movement and reconstruction of three-dimensional movement, for example.

Patent Document 2 discloses a method of forming an ultrasound probe with one-dimensional array transducers perpendicular to each other, and then trailing movement of the ultrasound probe. Specifically, the array directions of the respective array probes are the x-axis and the z-axis, respectively, and the beam direction is the y-axis. Amounts of movement in the x-axis and z-axis directions are calculated from images, to obtain a resultant vector. In this manner, a motion vector in the x-z plane is determined. An amount of movement in the y-axis direction is then calculated in one of the other two planes. As a result, a three-dimensional motion vector can be determined.

The method disclosed in Patent Document 2 is defined as a method of trailing movement of tissue (an object). However, it is difficult to detect rotation about the y-axis in the x-z plane by this method.

As for the x-axis and the z-axis, movement in the respective scanning planes can be detected by calculating affine parameters of the planes.

### CITATION LIST

### Patent Document

Patent Document 1: JP 2005-185333 A

Patent Document 2: JP 2010-227603 A

### SUMMARY OF THE INVENTION

### Problems to be Solved by the Invention

As described above, either of the conventional methods aims to calculate three-dimensional motion vectors of tissue, and is not compatible with rotation about the y-axis.

It is not likely that tissue has detectable rotational movement in the body. However, a motion vector of the probe needs to be calculated, and it is very likely that the probe rotates on the body surface. Therefore, detecting rotation about the y-axis is critical in calculating probe movement.

The present disclosure is made in view of those circumstances, and aims to improve the precision of probe movement calculations.

### Solutions to Problems

A signal processing apparatus of one aspect of the present disclosure includes: a probe including a first array transducer having a first scanning plane, and second array transducers each having a second scanning plane that intersects with the first scanning plane; and a signal processing unit that processes signals received from the probe or signals to be transmitted to the probe.

The number of transducers one-dimensionally arrayed in the first array transducer is larger than the number of transducers one-dimensionally arrayed in the second array transducers.

The second array transducers are located at both ends of the first array transducer.

The second scanning planes are perpendicular to the first scanning plane.

The signal processing apparatus may further include a control unit that controls a signal processing parameter of the signal processing unit.

The signal processing parameter is the frequencies of signals to be transmitted to the first array transducer and the second array transducers.

The control unit may control the frequencies of the signals to be transmitted to the first array transducer and the second array transducers so that the frequency of the signals to be transmitted to the second array transducers differs from the frequency of the signal to be transmitted to the first array transducer.

The signal processing parameter is the time to transmit signals to the first array transducer and the second array transducers.

The control unit controls the time to transmit signals to the first array transducer and the second array transducers so that a signal is transmitted to a transducer in the second array transducers, the transducer being located far from the transducer to which a signal is being transmitted among the transducers one-dimensionally arrayed in the first array transducer.

The signal processing parameter is a method for transmitting signals to the second array transducers.

The control unit may control the method for transmitting signals to the second array transducers so that signal transmission to the second array transducers is conducted with plane waves.

The signal processing parameter is switching on and off of transmission of signals to the second array transducers.

The control unit may control the switching on and off of the transmission of signals to the second array transducers so that the transmission of signals to the second array transducers is switched off.

A lens-shaped layer for beam focusing in a direction that intersects with the array direction of the first array transducer is provided on the first array transducer and the second array transducers at the side to be in contact with an object, the signal processing parameter is an amount of delay to be caused in the second array transducers by the lens-shaped layer, and the control unit may control the time to transmit signals to the second array transducers based on the amount of delay.

The signal processing apparatus may further include a movement calculation unit that calculates an amount of movement of the probe by using the signals processed by the signal processing unit.

The amount of movement of the probe is formed with an amount of movement in a plane in which the transducers constituting the first array transducer are one-dimensionally arrayed, and an angle of rotation about an axis perpendicular to the plane.

The movement calculation unit may reconstruct images by using the signals processed by the signal processing unit, and perform image matching to calculate the amount of movement of the probe.

The movement calculation unit may perform the image matching by calculating amounts of movement of intersection points in the first scanning plane, the intersection points being of the first scanning plane with respect to the second scanning planes.

The movement calculation unit may calculate the amount of movement of the probe by calculating phase variations of respective signals with the use of the signals processed by the signal processing unit.

A signal processing method of one aspect of the present disclosure includes processing signals received from a probe or signals to be transmitted to the probe, the processing being performed by a signal processing apparatus including a probe, the probe including: a first array transducer having a first scanning plane; and second array transducers each having a second scanning plane that intersects with the first scanning plane.

In one aspect of the present disclosure, signals received from a probe or signals to be transmitted to the probe are processed. The probe includes a first array transducer having a first scanning plane, and second array transducers each having a second scanning plane that intersects with the first scanning plane.

### Effects of the Invention

According to the present disclosure, the precision of probe movement calculations can be increased.

### BRIEF DESCRIPTION OF DRAWINGS

FIG. 1 is a block diagram showing an example structure of a conventional probe.

FIG. 2 is a diagram showing an example structure of a probe to which the present technique is applied.

FIG. 3 is a diagram for explaining imaging planes of array transducers.

FIG. 4 is a block diagram showing an example structure of a diagnostic ultrasound imaging apparatus to which the present technique is applied.

FIG. 5 is a block diagram showing a specific example structure of the diagnostic ultrasound imaging apparatus.

FIG. 6 is a diagram for explaining an acoustic lens in the probe.

FIG. 7 is a diagram for explaining the influence of an acoustic lens in the x-axis direction.

FIG. 8 is a diagram for explaining the influence of an acoustic lens in the z-axis direction.

FIG. 9 is a diagram for explaining a probe movement calculation in the diagnostic ultrasound imaging apparatus.

FIG. 10 is a diagram for explaining an application of the present technique to a two-dimensional array probe.

FIG. 11 is a flowchart for explaining ultrasound signal processing by the diagnostic ultrasound imaging apparatus.

FIG. 12 is a flowchart for explaining a probe movement calculation process.

FIG. 13 is a diagram for explaining transmission timing control on the respective array transducers in a probe.

FIG. 14 is a block diagram showing an example configuration of a computer.

### MODES FOR CARRYING OUT THE INVENTION

Modes for carrying out the present disclosure (hereinafter referred to as the embodiments) will be described below. Explanation will be made in the following order.

### 1. First Embodiment (Probe)

### 2. Second Embodiment (Diagnostic Ultrasound Imaging Apparatus)

### 3. Third Embodiment (BF Control Process)

### 4. Fourth Embodiment (Computer)

### First Embodiment

### Example Structure of a Conventional Probe

Referring to FIG. 1, a conventional probe is described for comparison before a probe according to the present technique is described.

A probe 11 shown in FIG. 1 is a linear probe with a one-dimensional array, for example. The probe 11 is a portion to be pressed against an object (a living object: skin, for example), and has an array transducer 21 formed with arranged transducers on the side to be in contact with an object. The transducers are ultrasound transducers, and have rectangular parallelepiped shapes. That is, the array transducer 21 is formed by arranging (arraying) transducers so that the short side of each transducer falls into line with the long side 11L of the probe 11.

In the example shown in FIG. 1, the y-axis indicates the direction of the main lobe of ultrasound that is output from the center of the array transducer 21 (or the center of the short side 11S of the probe 11). The x-axis is a direction parallel to the long side 11L of the probe 11 (or the transducer array direction), and indicates the linear scanning direction of the probe 11. Although not shown in the drawing, the z-axis indicates a direction parallel to the short side 11S of the probe 11 (or a direction perpendicular to the array direction). In the drawings hereafter, the x-axis, the y-axis, and the z-axis are defined in the same manner as in FIG. 1.

The lower side of the probe 11 (or the positive side of the y-axis) is the side to be in contact with an object, and a scanning plane 22 formed with scanning lines L1 through Ln is shown below the probe 11.

In the probe 11, to form the scanning line L1 that is the left scanning line in the drawing, an ultrasound beam B1 from the first through eighth transducers from the left of the array transducer 21 is emitted, for example. To form the scanning line L2 that is the next scanning line in the linear scanning direction, an ultrasound beam B2 from the second through ninth transducers from the left of the array transducer 21 is emitted, for example. To form the scanning line L3 that is the next scanning line in the linear scanning direction, an ultrasound beam B2 from the third through tenth transducers from the left of the array transducer 21 is emitted, for example.

The reflected wave of the emitted ultrasound beam B1 reflected by the object is received by the first through eighth transducers, which then subject the reflected wave to signal processing, to generate the scanning line L1. The reflected wave of the emitted beam B2 reflected by the object is received by the second through ninth transducers, which then subject the reflected wave to signal processing, to generate the scanning line L2. The reflected wave of the emitted beam B3 reflected by the object is received by the third through tenth transducers, which then subject the reflected wave to signal processing, to generate the scanning line L3.

As the transducers that emit ultrasound beams and receive reflected waves gradually shift in the linear scanning direction as described above, the probe 11 can reconstruct an image in the scanning plane 22 formed with the scanning lines L1 through Ln.

Although 13 transducers arrayed in the array transducer 21 are shown in the example in FIG. 1, those transducers are shown schematically, and the array transducer 21 is often formed with 64, 96, or 128 transducers, for example.

Also, in the example shown in FIG. 1, the probe 11 is formed with a linear probe. However, the present technique described below is not limited to a linear probe, and may be formed with a convex or sector probe, as long as the probe has a one-dimensional array.

### Example Structure of a Probe According to the Present Technique

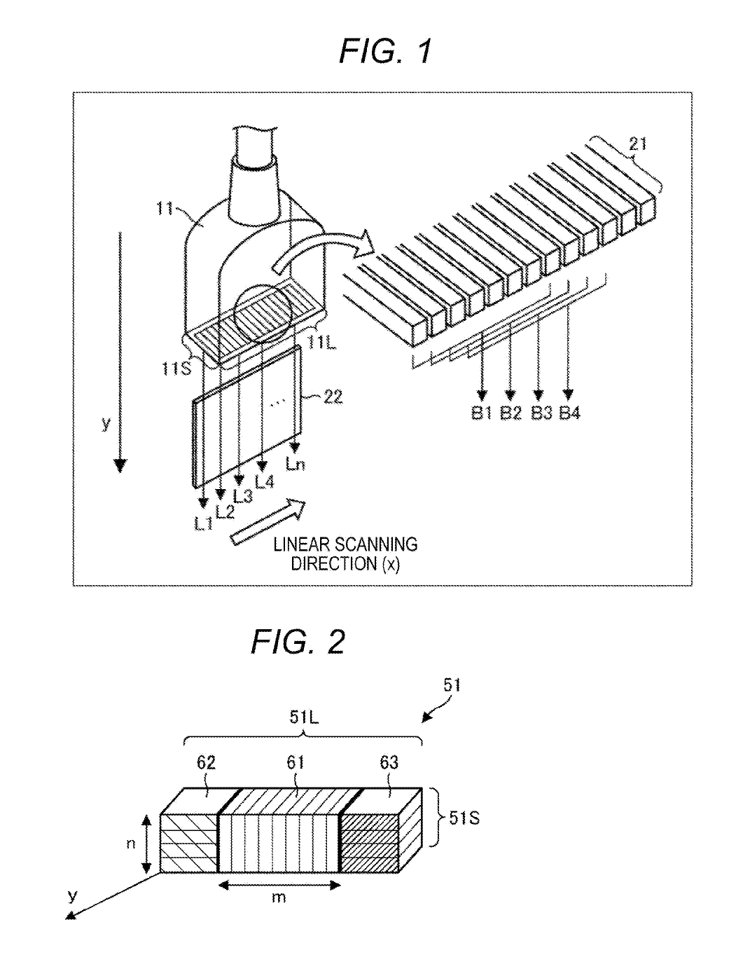

FIG. 2 is a diagram showing an example structure of a probe to which the present technique is applied.

The probe 51 shown in FIG. 2 is formed with an A array transducer 61, a B array transducer 62, and a C array transducer 63. Although only the array transducers constituting the probe 51 are shown in the example in FIG. 2, those array transducers are basically located in the same housing as that of the above described probe 11 shown in FIG. 1.

The A array transducer 61 is a one-dimensional array transducer equivalent to the array transducer 21 shown in FIG. 1. The B array transducer 62 and the C array transducer 63 are connected to both ends (the right and left ends in the drawing) of the A array transducer 61 in such a manner that the array direction of the respective transducers of the A array transducer 61 is perpendicular to the array direction of the respective transducers of the B array transducer 62 and the C array transducer 63.

Specifically, like the array transducer 21 in the probe 11 shown in FIG. 1, the respective transducers of the A array transducer 61 are arrayed in line with the long side 51L of the probe 51. On the other hand, the respective transducers of the B array transducer 62 and the C array transducer 63 are arrayed in line with the short side 51S of the probe 51.

As the B array transducer 62 and the C array transducer 63 are oriented in the direction of the tangent to rotation of the probe 51 in the above manner, the later described movement detection and rotation detection can be readily performed.

Here, the length of the long side 51L of the probe 51 includes (the length of the long side of each transducer of the B array transducer 62)+(the length of the A array transducer 61 in its array direction)+(the length of the long side of each transducer of the C array transducer 63). The length of the short side 51S of the probe 51 includes (the length of the long side of each transducer of the A array transducer 61) or (the length of the B array transducer 62 or the C array transducer 63 in its array direction).

The lengths of the B array transducer 62 and the C array transducer 63 are shorter than the length of the A array transducer 61 in its array direction. The shapes of the transducers constituting the respective array transducers are basically the same. That is, the numbers (n) of transducers arrayed in the B array transducer 62 and the C array transducer 63 are smaller than the number (m) of transducers arranged in the A array transducer 61.

As described above, the B array transducer 62 and the C array transducer 63 differ from the A array transducer 61 only in the number of arrayed transducers and the orientation in the probe 11, and the other aspects are basically the same as those of the A array transducer 61.

Although the numbers of transducers arrayed in the B array transducer 62 and the C array transducer 63 are both n in the example shown in FIG. 2, the numbers of transducers arrayed in the B array transducer 62 and the C array transducer 63 may differ from each other, as long as they are smaller than the number of transducers in the A array transducer 61.

Also, physical structures and properties, such as types, physicality, and filler, of the transducers constituting the probe 51 are not particularly limited.

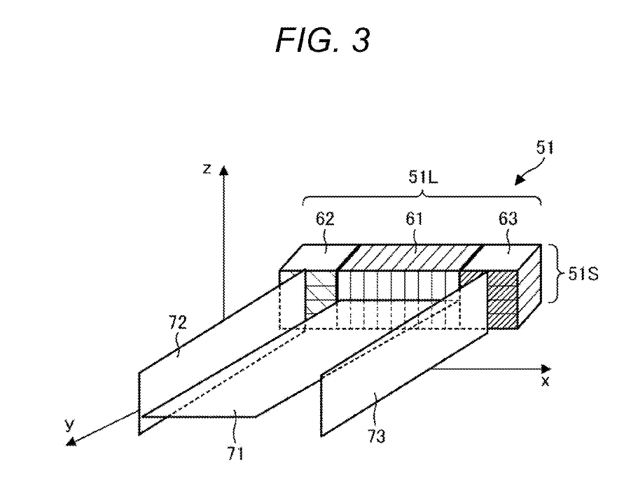

In the probe 51 having the above described structure, images can be reconstructed in three scanning planes as shown in FIG. 3.

### Example of the Imaging Planes of the Array Transducers

FIG. 3 is a diagram showing the imaging planes of the respective array transducers.

In the example shown in FIG. 3, the direction toward the right in the drawing is the positive direction of the x-axis, the direction toward the top is the positive direction of the z-axis, and the direction toward the left, the bottom, and the front is the positive direction of the y-axis. An A plane 71, a B plane 72, and a C plane 73 are perpendicular to the z-x plane formed by the x-axis extending in a direction parallel to the long side 51L of the probe 51 (the array direction of the A array transducer 61) and the z-axis extending in a direction parallel to the short side 51S of the probe 51 (the array direction of the B array transducer 62 and the C array transducer 63).

Specifically, the A plane 71 is an imaging plane reconstructed in a scanning plane that is located in the center of the long side of the transducers arrayed in the A array transducer 61, is parallel to the x-y plane, and is perpendicular to the z-x plane.

The B plane 72 is an imaging plane reconstructed in a scanning plane that is located in the center of the long side of the transducers arrayed in the B array transducer 62, is parallel to the y-z plane, and is perpendicular to the z-x plane.

The C plane 73 is an imaging plane reconstructed in a scanning plane that is located in the center of the long side of the transducers arrayed in the C array transducer 63, is parallel to the y-z plane, and is perpendicular to the x-z plane.

That is, the B plane 72 and the C plane 73 are planes parallel to each other, and are perpendicular to the A plane 71.

As described above, in the probe 51, the A array transducer 61, the B array transducer 62, and the C array transducer 63 are arranged so that the B plane 72 and the C plane 73 become parallel to each other and perpendicular to the A plane 71.

The probe 51 designed to form three scanning planes in the above manner will be hereinafter also referred to as a three-plane probe.

### Second Embodiment

### Example Structure of a Diagnostic Ultrasound Imaging Apparatus

Next, a diagnostic ultrasound imaging apparatus including the probe 51 described above with reference to FIGS. 2 and 3 is described.

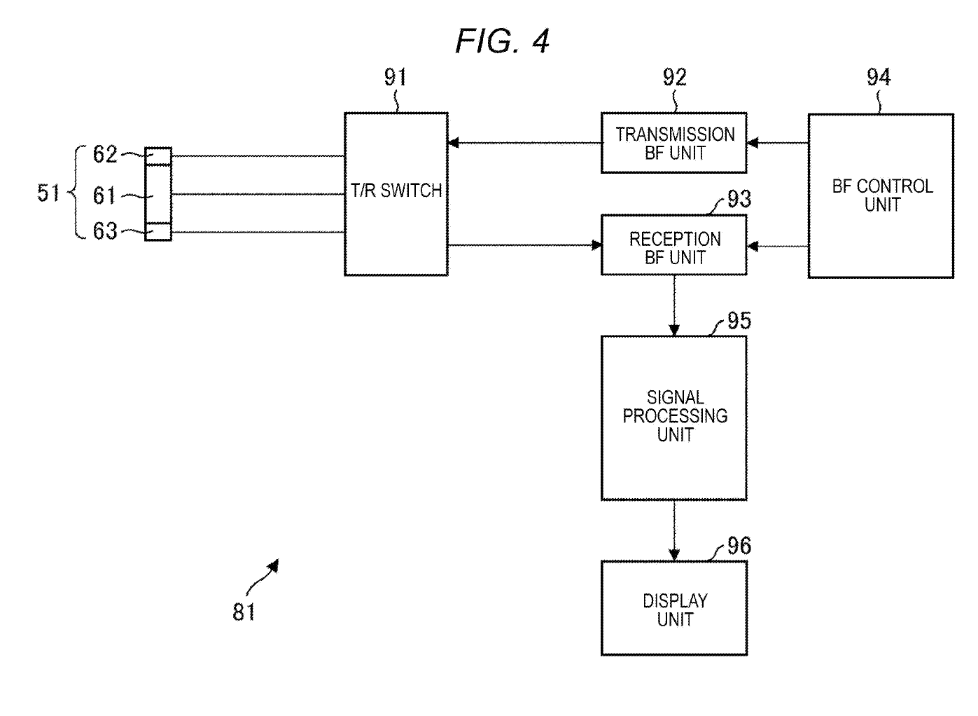

FIG. 4 is a block diagram showing an example structure of a diagnostic ultrasound imaging apparatus as a signal processing apparatus to which the present technique is applied.

The diagnostic ultrasound imaging apparatus 81 shown in FIG. 4 is an apparatus that includes the probe 51 described above with reference to FIGS. 2 and 3, captures an image (an ultrasound image) of the inside of an object by using ultrasound, and displays the image. The diagnostic ultrasound imaging apparatus 81 is used as a medical device for capturing images of the inside of the body of a patient or images of a fetus, or is used as an industrial device for capturing cross-sectional images of the inside of a product.

The diagnostic ultrasound imaging apparatus 81 is designed to include the probe 51, a T/R switch 91, a transmission BF (beam forming) unit 92, a reception BF unit 93, a BF control unit 94, a signal processing unit 95, and a display unit 96.

As described above with reference to FIGS. 2 and 3, the probe 51 is designed to include the A array transducer 61, the B array transducer 62, and the C array transducer 63.

The A array transducer 61, the B array transducer 62, and the C array transducer 63 transmit ultrasound beams to the object based on an ultrasound signal from the T/R switch 91. Meanwhile, the A array transducer 61, the B array transducer 62, and the C array transducer 63 receive reflected waves from the object, and supply the received signals to the T/R switch 91.

The T/R switch 91 is a switch for switching an ultrasound signal between transmission and reception. The T/R switch 91 receives an ultrasound signal from the transmission BF unit 92, and supplies the received ultrasound signal to the A array transducer 61, the B array transducer 62, or the C array transducer 63. The T/R switch 91 receives an ultrasound signal from the A array transducer 61, the B array transducer 62, or the C array transducer 63, and supplies the received ultrasound signal to the reception BF unit 93.

Under the control of the BF control unit 94, the transmission BF unit 92 performs a transmission beam forming process that is a process to generate an ultrasound signal (waveform), and supplies the signal subjected to the transmission beam forming process, to the T/R switch 91.

Under the control of the BF control unit 94, the reception BF unit 93 performs a reception beam forming process on a signal received from the T/R switch 91, and supplies the signal subjected to the reception beam forming process, to the signal processing unit 95.

Specifically, the reception beam forming process is a process to generate a reflected wave detection signal (hereinafter referred to as a RF signal) indicating the intensity of a reflected wave from a target point in the measurement field by adjusting the phases of received waves through a process of adding respective signals generated by delaying the received waves of the respective transducers (hereinafter referred to as the phase-adjusting addition process, where appropriate) based on the distances from the target point in the measurement field to the respective transducers in the probe 51.

The BF control unit 94 controls the transformation beam forming process of the transmission BF unit 92 and the reception beam forming process of the reception BF unit 93.

The ultrasound signal generated by the transmission beam forming process uniquely determines parameters such as the timing of beam emission from each array transducer (the transducers to be operated and the number of the transducers), the transmission frequency, and the transmission method.

In other words, the transmission BF unit 92 uniquely determines parameters such as the timing of beam emission from each array transducer (the transducers to be operated and the number of the transducers), the transmission frequency, and the transmission method, and generates an ultrasound signal in accordance with the combination of the determined parameters.

Therefore, the BF control unit 94 controls the transmission beam forming process of the transmission BF unit 92, to control (change) the signal processing parameters such as the timing of beam emission from each array transducer (the transducers to be operated and the number of the transducers), the transmission frequency, and the transmission method

The BF control unit 94 controls signal processing parameters in the reception beam forming process of the reception BF unit 93, such as the number of reception focusing points and the RF signal sampling frequency. The method of the signal processing parameter control by the BF control unit 94 will be described later.

The probe 51 shown in FIG. 4 can also be used as a one-dimensional array probe like the conventional probe 11 described above with reference to FIG. 1. In that case, the BF control unit 94 controls the transmission BF unit 92 to prohibit the transmission beam forming for the B array transducer 62 and the C array transducer 63.

As a result, the T/R switch 91 does not transmit a signal to the B array transducer 62 and the C array transducer 63, either. Accordingly, processing (such as a D/A conversion) of output ultrasound and an internal signal (not shown) is performed in the same manner as with a regular one-dimensional probe, and compatibility with the conventional probe 11 can be maintained. The compatibility means that, even if a user handles the probe 51 like the conventional probe 11, there will be no differences in operability and performance.

The signal processing unit 95 performs processing, mainly processing for imaging, on the RF signal generated by the reception BF unit 93, and supplies the imaged signal (or an image signal) to the display unit 96.

The display unit 96 displays the image corresponding to the image signal supplied from the signal processing unit 95.

In the example in FIG. 4, the functional blocks not directly related to the present technique are not shown.

### Specific Example Structure of the Diagnostic Ultrasound Imaging Apparatus

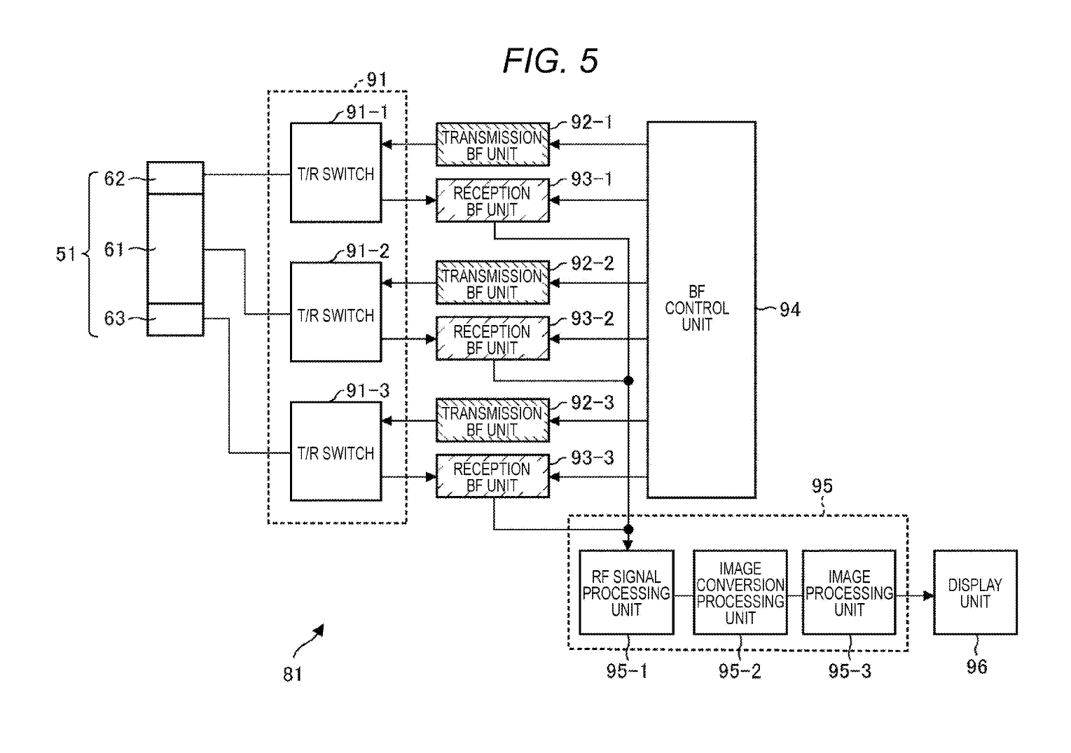

FIG. 5 shows a more specific example structure of the diagnostic ultrasound imaging apparatus shown in FIG. 4. In the example shown in FIG. 5, the blocks of transmission BF units 92-1 through 92-3 are hatched in the same shade. This means that those units are included in the transmission BF unit 92. Likewise, the blocks of reception BF units 93-1 through 93-3 are hatched in the same shade. This means that those units are included in the reception BF unit 93.

Specifically, in the example shown in FIG. 5, the T/R switch 91 is designed to include T/R switches 91-1 through 91-3. The transmission BF unit 92 is designed to include the transmission BF units 92-1 through 92-3. The reception BF unit 93 is designed to include the reception BF units 93-1 through 93-3. The signal processing unit 95 is designed to include a RF signal processing unit 95-1, an image conversion processing unit 95-2, and an image processing unit 95-3.

The T/R switch 91-1, the transmission BF unit 92-1, and the reception BF unit 93-1 correspond to the B array transducer 62. Specifically, the T/R switch 91-1 receives an ultrasound signal from the transmission BF unit 92-1, and supplies the received ultrasound signal to the B array transducer 62. The T/R switch 91-1 receives an ultrasound signal from the B array transducer 62, and supplies the received ultrasound signal to the reception BF unit 93-1.

Under the control of the BF control unit 94, the transmission BF unit 92-1 performs a transmission beam forming process that is a process to generate a signal (waveform) of an ultrasound beam emitted from the B array transducer 62, and supplies the signal subjected to the transmission beam forming process, to the T/R switch 91-1. Under the control of the BF control unit 94, the reception BF unit 93-1 performs a reception beam forming process on a signal received by the B array transducer 62 and transmitted from the T/R switch 91-1, and supplies the RF signal subjected to the reception beam forming process, to the RF signal processing unit 95-1.

The T/R switch 91-2, the transmission BF unit 92-2, and the reception BF unit 93-2 correspond to the A array transducer 61. Specifically, the T/R switch 91-2 receives an ultrasound signal from the transmission BF unit 92-2, and supplies the received ultrasound signal to the A array transducer 61. The T/R switch 91-2 receives an ultrasound signal from the A array transducer 61, and supplies the received ultrasound signal to the reception BF unit 93-2.

Under the control of the BF control unit 94, the transmission BF unit 92-2 performs a transmission beam forming process that is a process to generate a signal (waveform) of an ultrasound beam transmitted from the A array transducer 61, and supplies the signal subjected to the transmission beam forming process, to the T/R switch 91-2. Under the control of the BF control unit 94, the reception BF unit 93-2 performs a reception beam forming process on a signal received by the A array transducer 61 and transmitted from the T/R switch 91-2, and supplies the RF signal subjected to the reception beam forming process, to the RF signal processing unit 95-1.

The T/R switch 91-2, the transmission BF unit 92-2, and the reception BF unit 93-2 correspond to the A array transducer 61. Specifically, the T/R switch 91-2 receives an ultrasound signal from the transmission BF unit 92-2, and supplies the received ultrasound signal to the A array transducer 61. The T/R switch 91-2 receives an ultrasound signal from the A array transducer 61, and supplies the received ultrasound signal to the reception BF unit 93-2.

Under the control of the BF control unit 94, the transmission BF unit 92-3 performs a transmission beam forming process that is a process to generate a signal (waveform) of an ultrasound beam transmitted from the C array transducer 63, and supplies the signal subjected to the transmission beam forming process, to the T/R switch 91-3. Under the control of the BF control unit 94, the reception BF unit 93-3 performs a reception beam forming process on a signal received by the C array transducer 63 and transmitted from the T/R switch 91-3, and supplies the RF signal subjected to the reception beam forming process, to the RF signal processing unit 95-1.

The RF signal processing unit 95-1 performs signal processing on the RF signals from the reception BF units 93-1 through 93-3, and supplies the processed RF signals to the image conversion processing unit 95-2. The image conversion processing unit 95-2 performs a process to convert the RF signals from the RF signal processing unit 95-1 into image signals. The image conversion processing unit 95-2 supplies the converted image signals to the image processing unit 95-3.

The image processing unit 95-3 performs signal processing by using the image signals supplied from the image conversion processing unit 95-2. In one step in the signal processing, the image processing unit 95-3 calculates an amount of movement of the probe 51, to determine the amount of movement and the rotation angle of the probe 51. Based on the determined amount of movement and rotation angle of the probe 51, the image processing unit 95-3 generates an ultrasound image by turning the image into a panoramic image (with a wider viewing angle) and into volume data through image switching, and supplies the generated ultrasound image to the display unit 96.

[Acoustic Lens in the Probe]

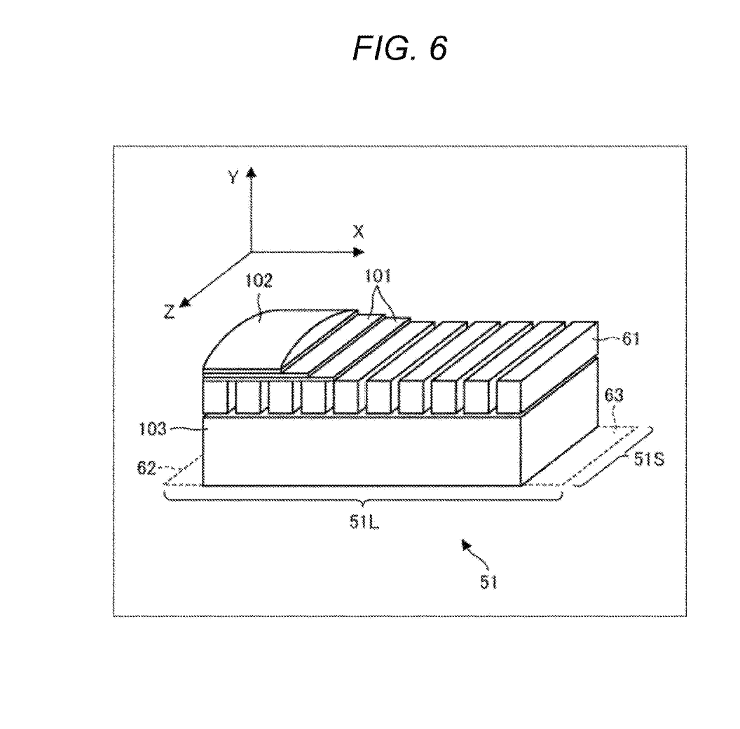

FIG. 6 shows an internal structure of the A array transducer 61 in the probe 51 at the side to be in contact with the object. In the example shown in FIG. 6, the direction toward the top is the positive direction of the y-axis, and indicates the side of the probe 51 to be in contact with the object. In the drawing, the direction toward the right is the positive direction of the x-axis, and the oblique direction toward the left is the positive direction of the z-axis.

The upper side of the A array transducer 61 shown in FIG. 6, or the side to be in contact with the object, has acoustic matching layers 101 stacked thereon, and an acoustic lens 102 is stacked on the acoustic matching layers 101. A packing material 103 is provided under the A array transducer 61. That is, the A array transducer 61 is stacked on the packing material 103.

The acoustic lens 102 has such a lens shape as to gather light along the short side 51S of the probe 51. With that shape, beam focusing in a direction (the z-axis direction) parallel to the short side 51S of the probe 51 is realized in the A array transducer 61. In the probe 51, an acoustic lens is also formed on each of the B array transducer 62 and the C array transducer 63 (dashed lines) provided at the right and left ends of the A array transducer 61, with this lens shape extending in the positive and negative directions of the x-axis.

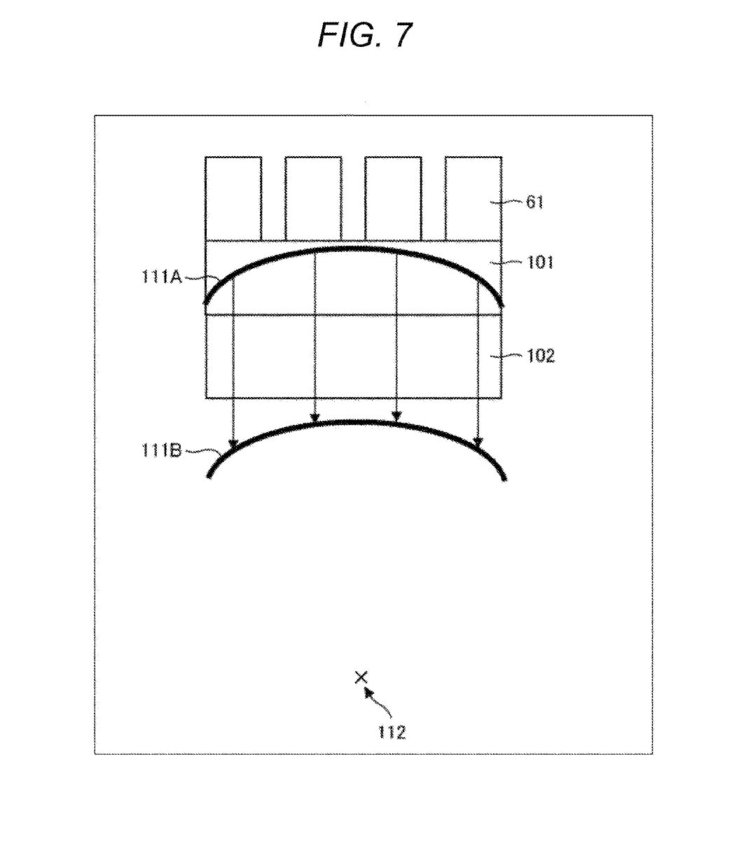

For example, the shape of the acoustic lens 102 in a cross-section taken from top to bottom (along the x-y plane) at the center of the short side 51S of the probe 51 shown in FIG. 6 is represented by a flat rectangle as shown in FIG. 7.

Therefore, in the x-axis direction beam forming of the A array transducer 61, a synthetic wave front 111A released from the A array transducer 61 is output as a synthetic wave front 111B shown in FIG. 7 from the acoustic lens 102, without a change in its shape. In such a case, the effect of the acoustic lens 102 can be ignored.

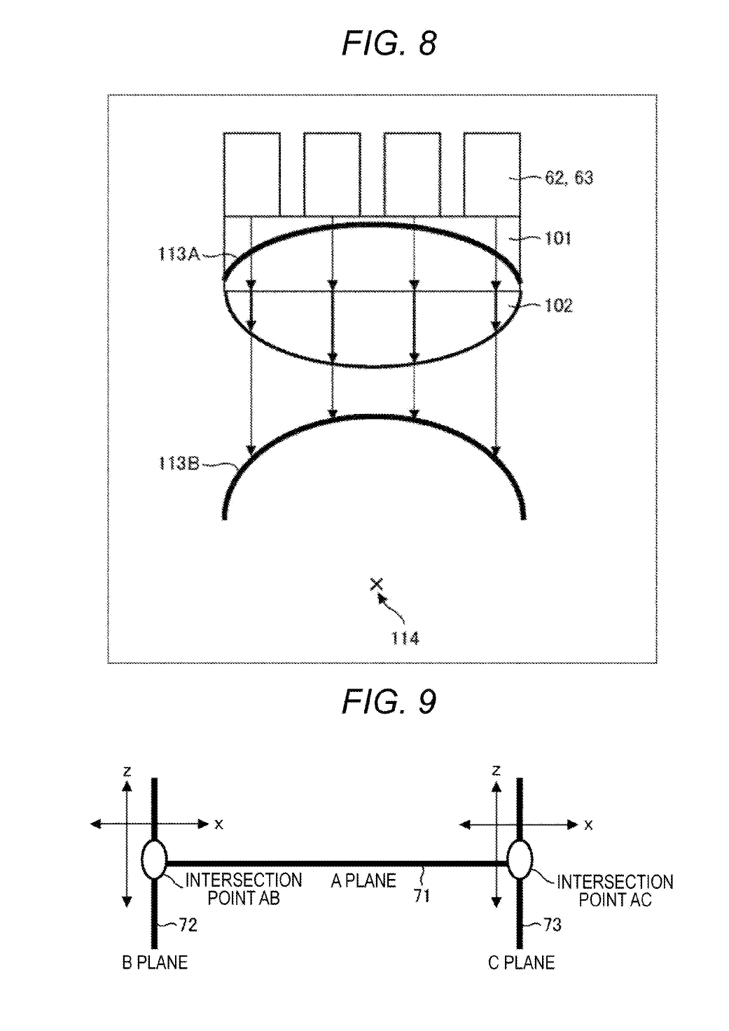

On the other hand, the acoustic lens 102 in a cross-section taken from top to bottom (along the y-z plane) at a site on the long side 51L of the probe 51 shown in FIG. 6 has a lens shape as shown in FIG. 8, for example. Therefore, in the z-axis direction beam forming of the B array transducer 62 and the C array transducer 63, a synthetic wave front 113A released from the B array transducer 62 and the C array transducer 63 is affected by the acoustic lens 102, like a synthetic wave front 113B shown in FIG. 8. Specifically, the synthetic wave front 113B changes to have a smaller R due to the lens effect of the acoustic lens 102, and a focal point 114 is formed in closer vicinity than a focal point 112 formed in the case with the synthetic wave front 111B shown in FIG. 7.

Therefore, when a beam is emitted from the B array transducer 62 or the C array transducer 63, a delay amount calculation or the like for beam forming needs to be performed, with the effect of the acoustic lens 102 being taken into account. However, only this difference needs to be taken into account in the delay amount calculation for beam forming, and an increase in the processing load in the actual delay amount calculation and a decrease in processing speed are not caused.

### Example of a Probe Movement Calculation Process

In a general coordinate transformation in a plane, there are degrees of freedom in parallel translation (in the x-direction and the y-direction), scaling, and rotation (about the y-axis). In a case where the contact area of the probe 51 moving on the surface of the body of a person, and the surface of the body of the person are regarded as flat surfaces, there is no need to take scaling into consideration, and only parallel translation (in the x-direction and the z-direction) and rotation (about the y-axis) need to be detected in practice.

When parallel translation parameters are calculated, it is necessary to know a movement (Δx, Δz) of at least one point. When a rotation angle is calculated, however, it is necessary to know movements of at least two points. As described above, by a detection method based on two planes perpendicular to each other as disclosed in Patent Document 2, only an amount of movement of one corresponding point can be calculated.

In the probe 51, on the other hand, the A plane 71, the B plane 72, and the C plane 73 are positioned so that two intersection points (an intersection point AB and an intersection point AC) are formed on the body surface, as shown in FIG. 9.

FIG. 9 shows the example arrangement of the A plane 71, the B plane 72, and the C plane 73 of FIG. 3 when seen from the y-axis direction. In the example shown in FIG. 9, the planes are arranged so that the B plane 72 and the C plane 73 become perpendicular to the A plane 71, and the intersection point AB between the A plane 71 and the B plane 72, and the intersection point AC between the A plane 71 and the C plane 73 are formed in the z-x plane.

Accordingly, the image processing unit 95-3 can calculate amounts of movement of the intersection point AB and the intersection point AC in the z-x plane, and then calculate an angle of rotation about the y-axis.

The example shown in FIG. 9 is a preferred example in which the A plane 71 is perpendicular to the B plane 72 and the C plane 73. However, the perpendicularity is not essential, as long as the A plane 71 intersects with (or is not parallel to) the B plane 72 and the C plane 73. Also, the B plane 72 and the C plane 73 are parallel to each other in the drawing, but may not be parallel to each other.

Using images reconstructed in the respective scanning planes (also referred to as B-mode images), the image processing unit 95-3 estimates an amount of movement of the probe 51. The method of estimating an amount of movement of the probe 51 is basically the same as a method of detecting a movement of an image. Specifically, between images reconstructed at time t and images reconstructed in the next frame t+Δt, amounts of movement of the intersection point AB and the intersection point AC in the entire imaging plane are calculated by a method such as feature point matching or block matching.

An ultrasound image is defined by the physical feature quantities of the probe 51 (such as the transducer pitch and the aperture size), the physical feature quantities of ultrasound (such as the frequency and the speed of sound), and the signal processing after reception (such as the frequency of an A-D conversion). Accordingly, an amount of movement (the number of pixels) in an image can be readily converted into an amount of movement (in a distance unit such as millimeter) in the actual body.

A reconstructed image in the A plane 71 is in the x-y plane, and reconstructed images in the B plane 72 and the C plane 73 are in the y-z plane. Among the obtained amounts of movement, the amounts of movement in the y-direction are not to be used in the later coordinate transformation parameter calculation. Specifically, (xt, zbt) and (xt+Δt, zbt+Δt) are calculated for the intersection point AB shown in FIG. 9, and (xt, zct) and (xt+Δt, zct+Δt) are calculated for the intersection point AC.

Those relations are applied to Helmert transformation formulas, and the formulas are then solved. In this manner, an amount of movement (x0, z0) and a rotation angle θ of the probe 51 can be calculated. The Helmert transformation formulas are expressed by the following equations (1).

x′=x cos θ−z sin θ+x0

z′=x sin θ+z cos θ+z0 (1)

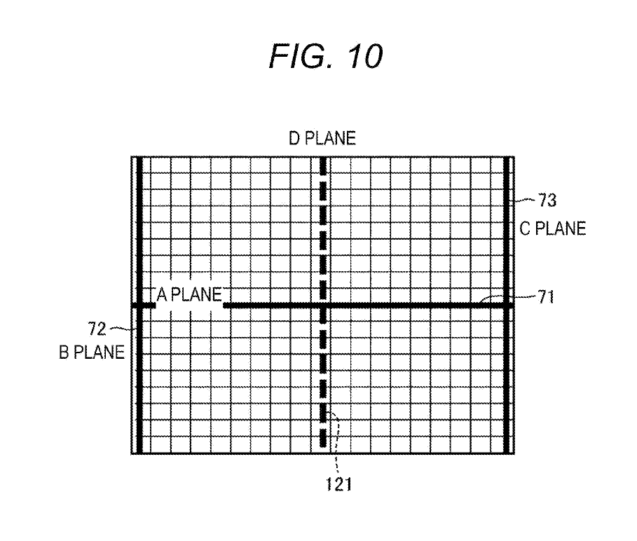

The above described movement calculation method can be applied in a case where a two-dimensional array probe formed with two-dimensionally arrayed transducers as shown in FIG. 10 is used. The respective squares shown in FIG. 10 represent transducers.

In a case where the method is applied to a two-dimensional array probe, the A plane 71, the B plane 72, and the C plane 73 may be formed as in the probe 51 of the present disclosure that has three scanning planes, or a D plane 121 indicated by the dashed line may be added between the B plane 72 and the C plane 73.

Also, the B plane 72, the C plane 73, and the D plane 121 are preferably perpendicular to the A plane 71 in the x-z plane. However, the above described movement calculation method can be applied, as long as those planes are not parallel to the A plane 71. The positional relations among the B plane 72, the C plane 73, and the D plane 121 shown in the example in FIG. 10 are merely an example, and those planes do not need to have the positional relations shown in FIG. 10. For example, the B plane 72 and the C plane 73 are preferably, but not necessarily, located at both ends of the detection range.

As described above, motion (movement parameters) of the probe 51 can be calculated with the probe 51 described above in the first embodiment and by the signal processing method that is implemented by the diagnostic ultrasound imaging apparatus 81 for the probe 51 as described above in the second embodiment.

In the above description, an amount of movement is calculated by performing image matching after images are reconstructed. However, an amount of movement may be estimated by performing signal processing on a RF signal prior to the reconstruction of images, and, based on the amount of movement, an amount of movement of the probe 51 may be calculated. In this case, the RF signal processing unit 95-1 performs the calculation process to calculate an amount of movement (or a phase variation in this case).

### Process to be Performed by the Diagnostic Ultrasound Imaging Apparatus

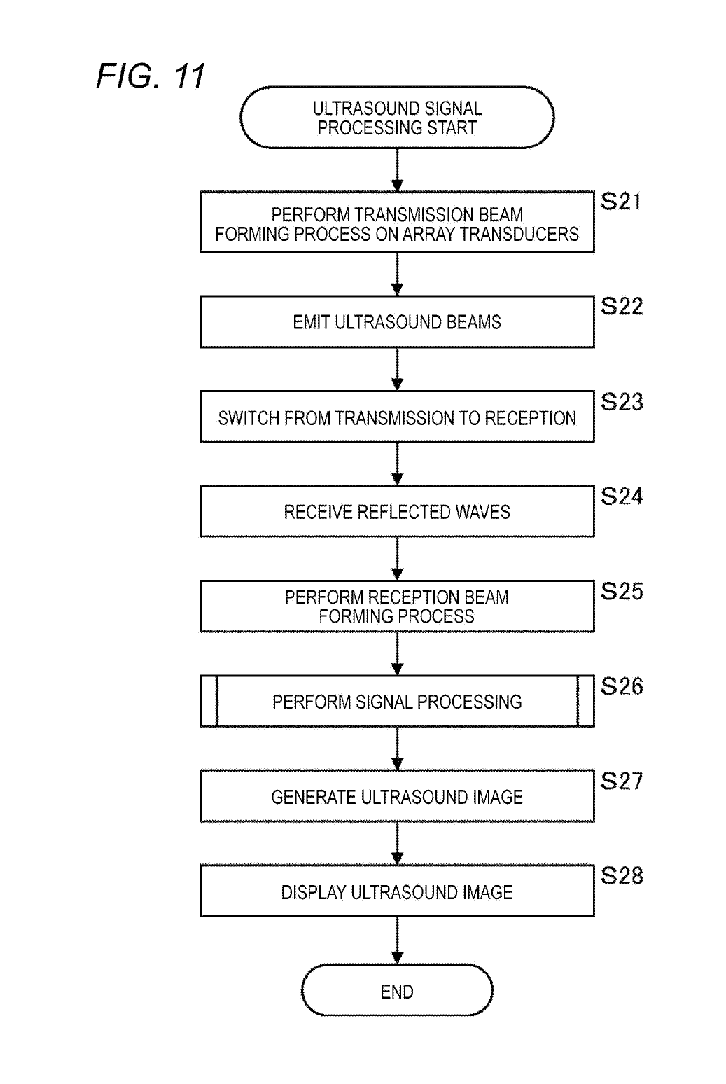

Referring now to the flowchart shown in FIG. 11, ultrasound signal processing by the diagnostic ultrasound imaging apparatus 81 is described.

In step S21, under the control of the BF control unit 94, the transmission BF unit 92 performs a transmission beam forming process on the A array transducer 61, the B array transducer 62, and the C array transducer 63, and supplies the signals subjected to the transmission beam forming process, to the T/R switch 91.

Specifically, under the control of the BF control unit 94, the transmission BF unit 92-1 performs a transmission beam forming process that is a process to generate a signal (waveform) of an ultrasound beam emitted from the B array transducer 62, and supplies the signal subjected to the transmission beam forming process, to the T/R switch 91-1. The T/R switch 91-1 receives the ultrasound signal from the transmission BF unit 92-1, and supplies the received ultrasound signal to the B array transducer 62.

Under the control of the BF control unit 94, the transmission BF unit 92-2 performs a transmission beam forming process that is a process to generate a signal (waveform) of an ultrasound beam transmitted from the A array transducer 61, and supplies the signal subjected to the transmission beam forming process, to the T/R switch 91-2. The T/R switch 91-2 receives the ultrasound signal from the transmission BF unit 92-2, and supplies the received ultrasound signal to the A array transducer 61.

Under the control of the BF control unit 94, the transmission BF unit 92-3 performs a transmission beam forming process that is a process to generate a signal (waveform) of an ultrasound beam transmitted from the C array transducer 63, and supplies the signal subjected to the transmission beam forming process, to the T/R switch 91-3. The T/R switch 91-3 receives the ultrasound signal from the transmission BF unit 92-3, and supplies the received ultrasound signal to the C array transducer 63.

In step S22, the A array transducer 61, the B array transducer 62, and the C array transducer 63 each emit an ultrasound beam to the object based on the ultrasound signal supplied from the T/R switch 91.

In step S23, the T/R switch 91 switches from transmission to reception by switching the position of an internal switch from the side of the transmission BF unit 92 to the side of the reception BF unit 93, for example.

Specifically, the T/R switch 91-1 switches from transmission to reception by switching the position of an internal switch from the side of the transmission BF unit 92-1 to the side of the reception BF unit 93-1, for example. The T/R switch 91-2 switches from transmission to reception by switching the position of an internal switch from the side of the transmission BF unit 92-2 to the side of the reception BF unit 93-2, for example. The T/R switch 91-3 switches from transmission to reception by switching the position of an internal switch from the side of the transmission BF unit 92-3 to the side of the reception BF unit 93-3, for example.

In step S24, the A array transducer 61, the B array transducer 62, and the C array transducer 63 receive the reflected waves corresponding to the ultrasound beams transmitted in step S22.

Specifically, the B array transducer 62 supplies the ultrasound signal corresponding to the received reflected wave, to the T/R switch 91-1. The T/R switch 91-1 receives an ultrasound signal from the B array transducer 62, and supplies the received ultrasound signal to the reception BF unit 93-1. The A array transducer 61 supplies the ultrasound signal corresponding to the received reflected wave, to the T/R switch 91-2. The T/R switch 91-2 receives an ultrasound signal from the A array transducer 61, and supplies the received ultrasound signal to the reception BF unit 93-2. The C array transducer 63 supplies the ultrasound signal corresponding to the received reflected wave, to the T/R switch 91-3. The T/R switch 91-3 receives the ultrasound signal from the C array transducer 63, and supplies the received ultrasound signal to the reception BF unit 93-3.

In step S25, under the control of the BF control unit 94, the reception BF unit 93 performs a reception beam forming process on the signals received from the T/R switch 91, and supplies the signals subjected to the reception beam forming process, to the signal processing unit 95.

Specifically, under the control of the BF control unit 94, the reception BF unit 93-1 performs a reception beam forming process on the signal received by the B array transducer 62 and transmitted from the T/R switch 91-1, and supplies the RF signal subjected to the reception beam forming process, to the signal processing unit 95. Under the control of the BF control unit 94, the reception BF unit 93-2 performs a reception beam forming process on the signal received by the A array transducer 61 and transmitted from the T/R switch 91-2, and supplies the RF signal subjected to the reception beam forming process, to the signal processing unit 95. Under the control of the BF control unit 94, the reception BF unit 93-3 performs a reception beam forming process on the signal received by the C array transducer 63 and transmitted from the T/R switch 91-3, and supplies the RF signal subjected to the reception beam forming process, to the signal processing unit 95.

In step S26, the signal processing unit 95 performs signal processing on the RF signals subjected to the reception beam forming process. Specifically, the RF signal processing unit 95-1 performs signal processing on the RF signals from the reception BF units 93-1 through 93-3, and supplies the processed RF signals to the image conversion processing unit 95-2. The image conversion processing unit 95-2 performs a process to convert the RF signals from the RF signal processing unit 95-1 into image signals. The image conversion processing unit 95-2 supplies the converted image signals to the image processing unit 95-3.

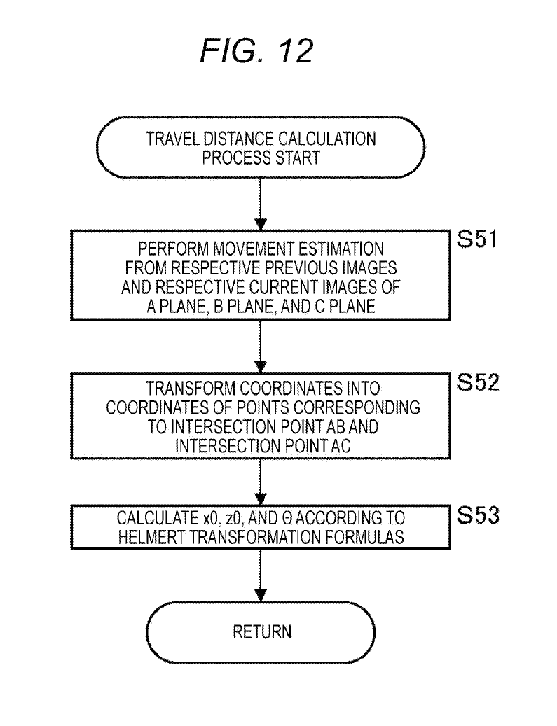

Using the image signals from the image conversion processing unit 95-2, the image processing unit 95-3 performs a process to calculate an amount of movement of the probe 51, as a step in the signal processing. The movement calculation process as a step in the signal processing performed on the probe 51 will be described later with reference to FIG. 12.

Through the process to calculate an amount of movement of the probe 51, an amount of movement of the probe 51 in the z-x plane and an angle of rotation of the probe 51 about the y-axis are calculated. In step S27, based on the amount of movement and the rotation angle determined in step S26, the image processing unit 95-3 generates an ultrasound image by turning the image into a panoramic image (with a wider viewing angle) and into volume data through image switching. The generated ultrasound image is supplied to the display unit 96.

In step S28, the display unit 96 displays the ultrasound image generated in step S27.

[Probe Movement Calculation Process]

Referring now to the flowchart shown in FIG. 12, the probe movement calculation process as a step in the signal processing in step S26 of FIG. 11 is described.

In step S51, the image processing unit 95-3 performs movement estimation by using the respective previous images and the respective current images of the A plane 71, the B plane 72, and the C plane 73. Specifically, between images reconstructed at time t and images reconstructed in the next frame t+Δt, the image processing unit 95-3 calculates amounts of movement of the intersection point AB and the intersection point AC in the entire imaging plane by using a method such as feature point matching or block matching.

In step S52, the image processing unit 95-3 transforms the coordinates of the intersection point AB and the intersection point AC in the image into the coordinates of the corresponding points in the actual living object.

In step S53, the image processing unit 95-3 calculates an amount of movement (x0, z0) and a rotation angle θ of the probe 51 according to the Helmert transformation formulas expressed by the equations (1). Specifically, the image processing unit 95-3 plugs the transformed coordinates into the Hermert transformation formulas expressed by the equations (1), and solves the formulas, to calculate the amount of movement (x0, z0) and the rotation angle θ of the probe 51.

As described above, motion (movement parameters) of the probe 51 can be calculated with the probe 51 that is a three-plane probe having three scanning planes, and by the signal processing method implemented by the diagnostic ultrasound imaging apparatus 81 for the probe 51.

### Third Embodiment

### Example of a BF Control Method

In the diagnostic ultrasound imaging apparatus 81, only the A plane 71 may be activated while the B plane 72 and the C plane 73 are not activated. That is, the probe 51 can also be used as a conventional one-dimensional array probe. Therefore, the probe 51 is sometimes used as a conventional one-dimensional array probe, and is sometimes used as a three-plane probe in which the B plane 72 and the C plane 73 are activated.

Between those two cases, it is not preferable to cause an image quality difference that will result in an error in diagnostic imaging.

With electronic scan of array probes being considered, an increase in the number of transducers normally leads to a decrease in the frame rate. That is, scanning the B plane 72 and the C plane 73 sacrifices the frame rate of the A plane 71.

In the diagnostic ultrasound imaging apparatus 81, only the image of the A plane 71 is used in diagnostic imaging. As described above with reference to FIG. 9, the B plane 72 and the C plane 73 are images to be used in calculating an amount of movement of the probe 51. Therefore, as long as an amount of movement can be calculated with sufficient precision, subjective image quality in the B plane 72 and the C plane 73 does not manner.

Based on the above concepts, the BF control unit 94 of the diagnostic ultrasound imaging apparatus 81 controls the signal processing parameters for signals to be transmitted to the A plane 71, the B plane 72, and the C plane 73, or ultrasound signals to be received from those planes.

Specifically, when the probe 51 is used as a three-plane probe, the BF control unit 94 controls transmission timing, transmission frequency, and beam forming, which are the signal processing parameters for ultrasound signal to be used in the transmission BF unit or the reception BF unit.

First, transmission timing control is described as a first signal processing parameter control method. The BF control unit 94 performs a synchronized operation to scan the B plane 72 and the C plane 73, without interrupting the operation to scan the A plane 71.

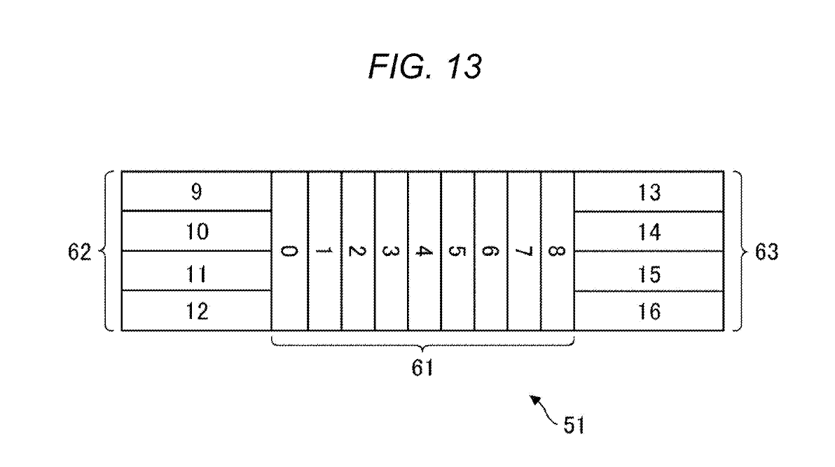

The BF control unit 94 scans a plane at the opposite end from the site being scanned in the A plane 71. That is, the BF control unit 94 simultaneously activates transducers that are physically far from each other. For example, the probe 51 has the structure shown in FIG. 13, and the aperture size in the scanning operation is equivalent to three elements.

As shown in the example in FIG. 13, the A array transducer 61 is designed to include transducers to which 0 through 8 are assigned from left to right. The B array transducer 62 located on the left side of the A array transducer 61 is designed to include transducers to which 9 through 12 are assigned from top to bottom. The C array transducer 63 located on the right side of the A array transducer 61 is designed to include transducers to which 13 through 12 are assigned from top to bottom. In practice, each of the array transducers is designed to include a larger number of transducers than those shown in FIG. 13.

In such a structure, the BF control unit 94 performs control so that a beam is also emitted from the transducers denoted by 13 and 14 in the C array transducer 63 when a beam is emitted from the transducers denoted by −1 (not shown), 0, and 1 in the A array transducer 61.

The BF control unit 94 performs control so that a beam is also emitted from the transducers denoted by 14 and 15 in the C array transducer 63 when a beam is emitted from the transducers denoted by 0, 1, and 2 in the A array transducer 61. The BF control unit 94 performs control so that a beam is also emitted from the transducers denoted by 15 and 16 in the C array transducer 63 when a beam is emitted from the transducers denoted by 1, 2, and 3 in the A array transducer 61. The BF control unit 94 performs control so that a beam is also emitted from the transducers denoted by 16 and 17 (not shown) in the C array transducer 63 when a beam is emitted from the transducers denoted by 2, 3, and 4 in the A array transducer 61.

The BF control unit 94 then performs control so that a beam is also emitted from the transducers denoted by 9 and 10 in the B array transducer 62 when a beam is emitted from the transducers denoted by 3, 4, and 5 in the A array transducer 61. The BF control unit 94 performs control so that a beam is also emitted from the transducers denoted by 10 and 11 in the B array transducer 62 when a beam is emitted from the transducers denoted by 4, 5, and 6 in the A array transducer 61. The BF control unit 94 performs control so that a beam is also emitted from the transducers denoted by 11 and 12 in the B array transducer 62 when a beam is emitted from the transducers denoted by 5, 6, and 7 in the A array transducer 61.

As described above, array transducers that are physically far from each other are activated at the same time, so that mutual interference is reduced, and a decrease in frame rate in a reconstructed image from the A array transducer 61 is prevented.

Next, transmission frequency control is described as a second signal processing parameter control method. For the A array transducer 61, an image formed in the A plane 71 is used in diagnostic imaging. Therefore, an ultrasound signal needs to be transmitted at a frequency suitable for the diagnosis purpose. This frequency affects the reachable depth and the frame rate.

Meanwhile, imaging is performed with respect to the B array transducer 62 and the C array transducer 63 located at the right and left ends of the A array transducer 61. However, the image formed in the B plane 72 and the image formed in the C plane 73 are to be used only in calculating an amount of movement, and are not to be used in diagnostic imaging. Therefore, a certain degree of freedom is allowed in setting the frequencies of ultrasound signals to be transmitted from the B array transducer 62 and the C array transducer 63.

Specifically, when the transmission frequency bandwidth for the A array transducer 61 is from 7.5 MHz to 10 MHz, frequency interference can be prevented by setting a higher transmission frequency bandwidth than 10 MHz for the B array transducer 62 and the C array transducer 63. Alternatively, the transmission frequency bandwidth for the B array transducer 62 and the C array transducer 63 may be set lower than 7.5 MHz. In that case, frequency interference can also be prevented.

Further, beam forming control is now described as a third signal processing parameter control method. The BF control unit 94 controls the B array transducer 62 and the C array transducer 63 to emit beams in the form of plane waves. That is, a technique involving electronic scan is used by the A array transducer 61 as usual, but a technique not involving electronic scan is used by the B array transducer 62 and the C array transducer 63.

As a result, the number of times a beam is transmitted from the B array transducer 62 and the C array transducer 63 is reduced. Accordingly, the signal separation by a distance according to the above described first control method becomes more effective.

Among the first through third signal processing parameter control methods described above, the most effective one is the second one, but all the first through third signal processing parameter control methods may be controlled. That is, the above mentioned various kinds of parameters may be controlled in a combined manner so as to dramatically reduce influence on the image quality in the A plane 71, and effectively increase the precision in calculating amounts of movement in the B plane 72 and the C plane 73.

It is not necessary to use all of or one of the first through third control methods, but any two of the first through third control methods may be used.

As described above, according to the present technique, the total number of elements can be made smaller than that in a conventional two-dimensional probe, and the production costs and the signal processing costs can be lowered accordingly.

For example, when the same image quality as the image quality achieved with a one-dimensional probe having 128 elements is to be achieved with a two-dimensional probe, the two-dimensional probe needs to have 128×128 elements. According to the present technique, on the other hand, the same image quality can be achieved with (128+16+16) elements, where the number of the elements in the direction perpendicular to the array direction, or the number of the elements in each of the B array transducer 62 and the C array transducer 63, is 16.

In the case of a 1.5-dimensional probe having 16 elements aligned in the perpendicular direction, the number of elements is 128×16. With the processing of the transducer material and the like being taken into account, the difference in the hardware cost between the present technique and a case where a 1.5-dimensional probe or a two-dimensional probe is manufactured is larger than the difference caused by the difference in the number of elements. That is, the hardware cost in the present technique can be maintained at a low level.

Also, according to the present technique, displacements of biaxial movements and uniaxial rotations can be measured as if the user were using a conventional one-dimensional probe. If the A array transducer 61 is formed with a small number of elements (96 elements, for example), the exterior of the probe 51 is exactly the same as a one-dimensional array probe formed with a large number of elements (128 elements, for example). Accordingly, when regular diagnostic imaging is performed without beam emission from the B array transducer 62 and the C array transducer 63, for example, the usability of the probe 51 does not differ from that of a conventional probe.

Also, when displacements are measured, it is not necessary to change probes. Actually, it is rare to use a two-dimensional probe as a one-dimensional probe. In view of this, the present technique is advantageous in terms of costs.

Furthermore, according to the present technique, decreases in the frame rate can be minimized. Specifically, images in the B plane 72 and the C plane 73, which intersect with the A plane 71, can be generated by controlling frequencies of beams and transmission/reception, without affecting the image quality in the A plane 71. Accordingly, even if an operation using the B plane 72 or the C plane 73 is being performed, the same situation as diagnostic imaging with conventional B-mode images can be reproduced.

Also, according to the present technique, movements of the probe 51 are detected with high precision. Accordingly, the precision of applications for position indications, panoramic images, and the like can be increased.

One of the principal objectives of acquisition of accurate probe position information is to obtain panoramic images (with a wider viewing angle) and volume data through image switching.

By a method using a conventional one-dimensional probe, high precision can be achieved in switching for movements in the long axis direction (the x-direction), but expansion in the short axis direction (the z-direction) is difficult. Also, a method of tilting the probe contact surface toward an axis so as to create volume data is now being put into practical use. In that case, however, the angle in doing so is fixed (there is an instruction to move the probe to a certain degree in a certain number of seconds), or a special system equipped with an angle sensor is used.

By a method using an angle sensor, volume data reproduction can be realized with a certain degree of accuracy. However, the contact surface of the probe does not move, and therefore, volume data of portions near the epidermis cannot be created.

According to the present technique, movements of a probe are detected with high precision. Accordingly, panoramic images (with a wider viewing angle) and volume data can be obtained more precisely through image switching.

The above described series of processes can be performed by hardware, and can also be performed by software. When the series of processes are to be performed by software, the programs forming the software are installed into a computer. Note that examples of the computer include a computer embedded in dedicated hardware and a general-purpose personal computer capable of executing various functions by installing various programs therein.

### Fourth Embodiment

### Example Configuration of a Computer

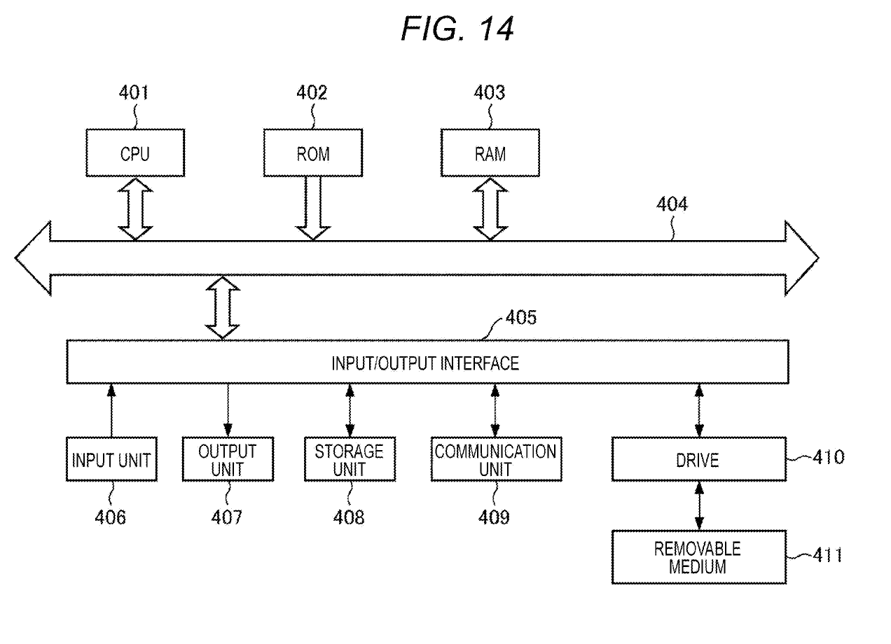

FIG. 14 is a block diagram showing an example configuration of the hardware of a computer that performs the above described series of processes in accordance with programs.

In the computer, a CPU (Central Processing Unit) 401, a ROM (Read Only Memory) 402, and a RAM (Random Access Memory) 403 are connected to one another by a bus 404.

An input/output interface 405 is further connected to the bus 404. An input unit 406, an output unit 407, a storage unit 408, a communication unit 409, and a drive 410 are connected to the input/output interface 405.

The input unit 406 is formed with a keyboard, a mouse, a microphone, and the like. The output unit 407 is formed with a display, a speaker, and the like. The storage unit 408 is formed with a hard disk, a nonvolatile memory, or the like. The communication unit 409 is formed with a network interface or the like. The drive 410 drives a removable medium 411 such as a magnetic disk, an optical disk, a magnetooptical disk, or a semiconductor memory.

In the computer having the above described configuration, the CPU 401 loads a program from the storage unit 408 into the RAM 403 via the input/output interface 405 and the bus 404, and executes the program to perform the above described series of processes.

The programs to be executed by the computer (the CPU 401) may be recorded on the removable medium 411 as a package medium to be provided, for example. Alternatively, the programs may be provided via a wired or wireless transmission medium, such as a local area network, the Internet, or digital broadcasting.

In the computer, the programs can be installed into the storage unit 408 via the input/output interface 405 when the removable medium 411 is mounted on the drive 410. Also, the programs may be received by the communication unit 409 via a wired or wireless transmission medium, and be installed into the storage unit 408. Other than that, the program can be installed beforehand into the ROM 402 or the storage unit 408.

The programs to be executed by the computer may be programs for performing processes in chronological order in accordance with the sequence described in this specification, or may be programs for performing processes in parallel or performing a process when necessary, such as when there is a call.

In this specification, the term “system” means an entire apparatus formed with devices, blocks, and means.

Embodiments of the present disclosure are not limited to the above described embodiments, and various changes may be made to them without departing from the scope of the present disclosure.

Although preferred embodiments of the present disclosure have been described with reference to the accompanying drawings, the present disclosure is not limited to those examples. It is obvious that a person with ordinary knowledge of the technical field of the present disclosure can think of various changes and modifications within the technical ideas claimed herein, and those changes and modification are of course considered to be included in the technical scope of the present disclosure.

The present technique can also be in the following forms.

(1) A signal processing apparatus including:

a probe that includes:
a first array transducer having a first scanning plane; and
second array transducers each having a second scanning plane that intersects with the first scanning plane; and

a signal processing unit that processes signals received from the probe or signals to be transmitted to the probe.

(2) The signal processing apparatus of (1), wherein the number of transducers one-dimensionally arrayed in the first array transducer is larger than the number of transducers one-dimensionally arrayed in the second array transducers.

(3) The signal processing apparatus of (1) or (2), wherein the second array transducers are located at both ends of the first array transducer.

(4) The signal processing apparatus of any of (1) through (3), wherein the second scanning planes are perpendicular to the first scanning plane.

(5) The signal processing apparatus of any of (1) through (4), further including a control unit that controls a signal processing parameter of the signal processing unit.

(6) The signal processing apparatus of (5), wherein the signal processing parameter is the frequencies of signals to be transmitted to the first array transducer and the second array transducers.

(7) The signal processing apparatus of (6), wherein the control unit controls the frequencies of the signals to be transmitted to the first array transducer and the second array transducers so that the frequency of the signals to be transmitted to the second array transducers differs from the frequency of the signal to be transmitted to the first array transducer.

(8) The signal processing apparatus of (5), wherein the signal processing parameter is the time to transmit signals to the first array transducer and the second array transducers

(9) The signal processing apparatus of (8), wherein the control unit controls the time to transmit signals to the first array transducer and the second array transducers so that a signal is transmitted to a transducer in the second array transducers, the transducer being located far from the transducer to which a signal is being transmitted among the transducers one-dimensionally arrayed in the first array transducer.

(10) The signal processing apparatus of (9), wherein the signal processing parameter is a method for transmitting signals to the second array transducers.

(11) The signal processing apparatus of (10), wherein the control unit controls the method for transmitting signals to the second array transducers so that signal transmission to the second array transducers is conducted with plane waves.

(12) The signal processing apparatus of (5), wherein the signal processing parameter is switching on and off of transmission of signals to the second array transducers.

(13) The signal processing apparatus of (12), wherein the control unit controls the switching on and off of the transmission of signals to the second array transducers so that the transmission of signals to the second array transducers is switched off.

(14) The signal processing apparatus of (5), wherein

a lens-shaped layer for beam focusing in a direction that intersects with the array direction of the first array transducer is provided on the first array transducer and the second array transducers at the side to be in contact with an object,

the signal processing parameter is an amount of delay to be caused in the second array transducers by the lens-shaped layer, and

the control unit controls the time to transmit signals to the second array transducers based on the amount of delay.

(15) The signal processing apparatus of any of (1) through (14), further including a movement calculation unit that calculates an amount of movement of the probe by using the signals processed by the signal processing unit.

(16) The signal processing apparatus of (15), wherein the amount of movement of the probe is formed with an amount of movement in a plane in which the transducers constituting the first array transducer are one-dimensionally arrayed, and an angle of rotation about an axis perpendicular to the plane.

(17) The signal processing apparatus of (15), wherein the movement calculation unit reconstructs images by using the signals processed by the signal processing unit, and performs image matching to calculate the amount of movement of the probe.

(18) The signal processing apparatus of (17), wherein the movement calculation unit performs the image matching by calculating amounts of movement of intersection points in the first scanning plane, the intersection points being of the first scanning plane with respect to the second scanning planes.

(19) The signal processing apparatus of (15), wherein the movement calculation unit calculates the amount of movement of the probe by calculating phase variations of respective signals with the use of the signals processed by the signal processing unit.

(20) A signal processing method including

processing signals received from a probe or signals to be transmitted to the probe,

the processing being performed by a signal processing apparatus including a probe,

the probe including:
a first array transducer having a first scanning plane; and
second array transducers each having a second scanning plane that intersects with the first scanning plane.

### REFERENCE SIGNS LIST

51 Probe
61 A array transducer
62 B array transducer
63 C array transducer
71 A plane
72 B plane
73 C plane
81 Diagnostic ultrasound imaging apparatus
91, 91-1 to 91-3 T/R switch
92, 92-1 to 92-3 Transmission BF unit
93, 93-1 to 93-3 Reception BF unit
94 BF control unit
95 Signal processing unit
95-1 RF signal processing unit
95-2 Image conversion processing unit
95-3 Image processing unit
96 Display unit
101 Acoustic matching layer
102 Acoustic lens
103 Packing material
111A, 111B Synthetic wave front
112 Focal point
113A, 113B Synthetic wave front
114 Focal point
121 D plane

1. A signal processing apparatus comprising:
a probe including:
a first array transducer having a first scanning plane; and
a plurality of second array transducers each having a second scanning plane that intersects with the first scanning plane; and
a signal processing unit configured to process signals received from the probe or signals to be transmitted to the probe.

2. The signal processing apparatus according to claim 1, wherein the number of transducers one-dimensionally arrayed in the first array transducer is larger than the number of transducers one-dimensionally arrayed in the second array transducers.

3. The signal processing apparatus according to claim 2, wherein the second array transducers are located at both ends of the first array transducer.

4. The signal processing apparatus according to claim 2, wherein the second scanning planes are perpendicular to the first scanning plane.

5. The signal processing apparatus according to claim 2, further comprising
a control unit configured to control a signal processing parameter of the signal processing unit.

6. The signal processing apparatus according to claim 5, wherein the signal processing parameter is frequencies of signals to be transmitted to the first array transducer and the second array transducers.

7. The signal processing apparatus according to claim 6, wherein the control unit controls the frequencies of the signals to be transmitted to the first array transducer and the second array transducers so that a frequency of signals to be transmitted to the second array transducers differs from a frequency of a signal to be transmitted to the first array transducer.

8. The signal processing apparatus according to claim 5, wherein the signal processing parameter is a time to transmit signals to the first array transducer and the second array transducers.

9. The signal processing apparatus according to claim 8, wherein the control unit controls the time to transmit signals to the first array transducer and the second array transducers so that a signal is transmitted to a transducer in the second array transducers, the transducer being located far from a transducer to which a signal is being transmitted among the transducers one-dimensionally arrayed in the first array transducer.

10. The signal processing apparatus according to claim 5, wherein the signal processing parameter is a method for transmitting signals to the second array transducers.

11. The signal processing apparatus according to claim 10, wherein the control unit controls the method for transmitting signals to the second array transducers so that signal transmission to the second array transducers is conducted with plane waves.

12. The signal processing apparatus according to claim 5, wherein the signal processing parameter is switching on and off of transmission of signals to the second array transducers.

13. The signal processing apparatus according to claim 12, wherein the control unit controls the switching on and off of the transmission of signals to the second array transducers so that the transmission of signals to the second array transducers is switched off.

14. The signal processing apparatus according to claim 2, wherein
a lens-shaped layer for beam focusing in a direction that intersects with the array direction of the first array transducer is provided on the first array transducer and the second array transducers at a side to be in contact with an object,
the signal processing parameter is an amount of delay to be caused in the second array transducers by the lens-shaped layer, and
the control unit controls a time to transmit signals to the second array transducers based on the amount of delay.

15. The signal processing apparatus according to claim 1, further comprising
a movement calculation unit configured to calculate an amount of movement of the probe by using the signals processed by the signal processing unit.

16. The signal processing apparatus according to claim 15, wherein the amount of movement of the probe is formed with an amount of movement in a plane in which the transducers constituting the first array transducer are one-dimensionally arrayed, and an angle of rotation about an axis perpendicular to the plane.

17. The signal processing apparatus according to claim 15, wherein the movement calculation unit reconstructs images by using the signals processed by the signal processing unit, and performs image matching to calculate the amount of movement of the probe.

18. The signal processing apparatus according to claim 17, wherein the movement calculation unit performs the image matching by calculating amounts of movement of intersection points in the first scanning plane, the intersection points being of the first scanning plane with respect to the second scanning planes.

19. The signal processing apparatus according to claim 15, wherein the movement calculation unit calculates the amount of movement of the probe by calculating phase variations of respective signals, using the signals processed by the signal processing unit.

20. A signal processing method comprising
processing signals received from a probe or signals to be transmitted to the probe,
the processing being performed by a signal processing apparatus including the probe,
the probe including:
a first array transducer having a first scanning plane; and
a plurality of second array transducers each having a second scanning plane that intersects with the first scanning plane.
